(19) United States
(12) Patent Application Publication
(10) Pub. No.: US 2025/0072470 A1
(43) Pub. Date: Mar. 6, 2025

(54) METHOD OF PRODUCTION OF A VAPING LIQUID

(71) Applicants: Shaun Theo Devries, Victoria (CA); Jaymes Thomas Scott-Polson George, Malahat (CA)

(72) Inventors: Shaun Theo Devries, Victoria (CA); Jaymes Thomas Scott-Polson George, Malahat (CA)

(21) Appl. No.: 18/459,022

(22) Filed: Aug. 30, 2023

(51) Int. Cl. A24B 15/167; A24B 15/22; A24B 15/30; A24B 15/32; A61K 9/08; A61K 31/00; A61K 47/10

(52) U.S. Cl. CPC A24B 15/167; A24B 15/22; A24B 15/303; A24B 15/32; A61K 9/08; A61K 31/658; A61K 47/10

(57) ABSTRACT

A method operable to produce an infused liquid configured to be utilized in electronic cigarettes wherein the infused liquid includes an active ingredient compound selected from cannabis, hemp extract, plant extract or fungi extract. The present invention produces a formulation that maximizes the potency of the active ingredient of the formulation. The present invention includes a decarboxylation process facilitated via heat exposure wherein a mixture of a carrier base and the active ingredient is placed in a container and heat and agitation are applied thereto. The method of the present invention further includes exposure of the mixture of a carrier base and the active ingredient to an ultrasonic frequency of at least 20 KHZ. The mixture of the mixture of a carrier base and the active ingredient is exposed to ultrasonic frequencies for alternate time periods dependent upon the active ingredient compound mixed selected to be mixed with the carrier base.

### FIELD OF THE INVENTION

The present invention relates generally to vaping liquids, more specifically but not by way of limitation, a method operable to produce an infused liquid with an active ingredient such as but not limited to cannabis, wherein the infused liquid is configured to be consumed via an electronic cigarette.

### BACKGROUND

Electronic cigarettes, also known as vaping, has exploded in popularity as a substitute for conventional cigarettes. As is known in the art, electronic cigarettes contain a power source, a heating element and a liquid reservoir, typically referred to as a cartridge. The conventional electronic cigarette employs a liquid that contains nicotine and a flavoring agent wherein the liquid is vaporized by the heating element and is inhaled by the user. Most cartridges are one-time use and as such once the liquid is consumed, the cartridge is discarded. It is quite common for the cartridges to leak and as such result in the loss of the liquid available to be vaporized and consumed.

Cannabis has become legal in many countries and states and is now consumed by millions for recreational and health benefits. Infusion of cannabis into liquids for electronic cigarettes is minimally available from a commercial perspective due to the intrinsic challenges associated with the process. Another issue that is common with cartridge tips is the clogging thereof. It is quite common for one milliliter vaping tips to have crystallization issues which results in the clogging thereof. Furthermore, the desire from an environmental and user perspective for a refillable electronic cigarette cartridge is desirable and would result in a significant waste reduction.

Accordingly, there is a need for a cannabis infused electronic cigarette liquid that is operable to be deposited into refillable cartridges wherein the present invention provides a electronic cigarette liquid that is infused with a cannabis or hemp extracted compound.

### SUMMARY OF THE INVENTION

It is the object of the present invention to provide a method for producing a cannabis infused liquid suitable for utilization in an electronic cigarette wherein the present invention initiates with a carrier base of polyethylene glycol.

Another object of the present invention is to provide a cannabis infused vaping liquid that is manufactured to be consumed through utilization of an electronic cigarette wherein the present invention includes addition of a cannabis or hemp extracted compound to the carrier base such as but not limited to THC and all derivatives thereof, CBD and all derivatives thereof as well as alternate plant extracts.

A further object of the present invention is to provide a method for producing a cannabis infused liquid suitable for utilization in an electronic cigarette that includes a step of combining the carrier base and the cannabis compound in an appropriate vessel and applying heat thereto.

Yet a further object of the present invention is to provide a cannabis infused vaping liquid that is manufactured to be consumed through utilization of an electronic cigarette that further employs the step of utilizing an ultrasonic bath on the mixture of the carrier base and cannabis compound.

Still another object of the present invention is to provide a method for producing a cannabis infused liquid suitable for utilization in an electronic cigarette wherein the exposure to ultrasonic frequencies range from three to thirty minutes.

An additional object of the present invention is to provide a cannabis infused vaping liquid that is manufactured to be consumed through utilization of an electronic cigarette wherein the ultrasonic frequency employed is at least 20 kilohertz.

Yet a further object of the present invention is to provide a method for producing a cannabis infused liquid suitable for utilization in an electronic cigarette wherein the step of combining the carrier base and the cannabis employs a double boiler apparatus and a temperature of two hundred to two hundred and forty five degrees Fahrenheit.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a flowchart of the method of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated a vaping liquid manufacturing method 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

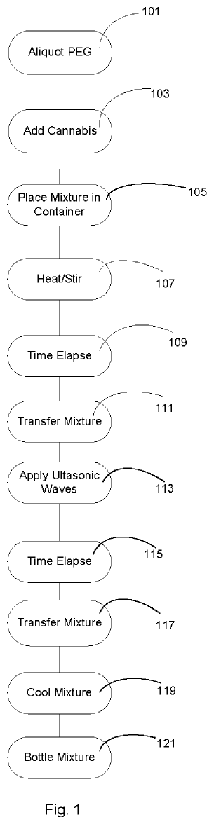

Referring in particular to the Figures submitted herewith, the vaping liquid manufacturing method 100 is diagrammed in FIG. 1 herein. It should be understood within the scope of the present invention that while a particular order has been illustrated and discussed herein that it is contemplated within the scope of the present invention that the steps could be executed in alternate order, be executed in combination or be eliminated. In step 101, a carrier base is aliquoted from a supply wherein the carrier base is placed in a suitable container. While various carrier bases can be employed for the present invention, in a preferred embodiment of the present invention the carrier base is a mixture of propylene glycol and vegetable glycerin. In step 103, a desired amount of active ingredient compound is added to the carrier base mixture. It should be understood within the scope of the present invention that the active ingredient compound is selected from a group comprising of: cannabis, hemp extract, plant extract or fungi extract. As it pertains to cannabis and hemp extracts this is to include but not be limited to the following compounds: THC,THCV,THCA,CBD,CBT,CBTC,CBCa,CBC,CNB,CBNA,CBDA,CBG,CBGA,HHC,CBDVA, CBGVA,CBDV and CBCV.

Step 105, the mixture of the carrier base and active ingredient compound is placed into a container suitable for heating thereof. In a preferred embodiment of the present invention the mixture of the carrier base and active ingredient compound is placed in a laboratory grade double boiler for controlled heating and stirring thereof. In step 107, the mixture of the carrier base and active ingredient compound disposed in the double boiler is heated to a temperature between two hundred and two hundred and forty five degrees Fahrenheit. During the heating of the mixture of the carrier base and active ingredient compound constant stirring additionally occurs. Step 107 is a decarboxylation process that provides maximization of the potency of the extracted active ingredient by breaking down the cellular wall of the active ingredient allowing the carrier base to bind therewith.

In step 109, the mixture of the carrier base and active ingredient compound is heated and stirred at the aforementioned temperature range for a time ranging from five to one hundred and twenty minutes. The time that the mixture of the carrier base and active ingredient compound is exposed to heat and agitation depends upon the active ingredient compound that is being employed. Step 111, ensuing exposure to heat and agitation, the mixture of the carrier base and active ingredient compound is transferred to a suitable container. In step 113, the container having the mixture of the carrier base and active ingredient compound disposed therein is submitted to ultrasonic waves. In a preferred embodiment of the present invention the mixture of the carrier base and active ingredient compound is exposed to at least 20 KHZ frequency. Employing the aforementioned frequency ensures penetration through the mixture of the carrier base and active ingredient compound disposed in the container. Step 115, the mixture of the carrier base and active ingredient compound is exposed to the ultrasonic waves for a time period ranging between three and thirty minutes. The time period employed is dependent upon the active ingredient compound selected.

In step 117, ensuing exposure to the ultrasonic waves, the mixture of the carrier base and active ingredient compound is transferred to a suitable container. Step 119, the mixture of the carrier base and active ingredient compound is cooled to an ambient temperature. Cooling can be employed either at room temperature or be assisted with utilization of refrigeration to achieve an ambient temperature foe the mixture of the carrier base and active ingredient compound. In step 121, the mixture of the carrier base and active ingredient compound is placed into suitable containers for sale thereof. It should be understood within the scope of the present invention that the mixture of the carrier base and active ingredient compound could be placed in alternate sizes of containers for sale thereof.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A method operable to produce a liquid suitable for utilization in an electronic cigarette wherein the method comprises the steps of:
selecting a desired amount of a carrier base, wherein said carrier base is placed in a suitable vessel;
adding an active ingredient compound, wherein an amount of the active ingredient compound is added to the carrier base in the vessel;
applying heat and agitation to the mixture of the carrier base and active ingredient disposed in the vessel;
allowing a required amount of time for the mixture to have heat and agitation applied thereto;
transferring the mixture to a second vessel;
applying ultrasonic frequency to the second vessel having the mixture of the carrier base and active ingredient disposed therein;
permitting a required amount of time to elapse for exposure of the ultrasonic frequency to the second vessel;
transferring the mixture of the carrier base and active ingredient to a third vessel;
cooling the mixture of the carrier base and active ingredient in the third vessel.

2. The method operable to produce the liquid suitable for utilization in an electronic cigarette as recited in claim 1, wherein the active ingredient is selected from a group consisting of: cannabis, hemp extract, plant extract or fungi extract.

3. The method operable to produce the liquid suitable for utilization in an electronic cigarette as recited in claim 2, wherein the step of applying an ultrasonic frequency utilizes an ultrasonic frequency of at least 20 KHZ.

4. The method operable to produce the liquid suitable for utilization in an electronic cigarette as recited in claim 3, wherein the step of applying heat to the the mixture of the carrier base and active ingredient is performed at a temperature range between 200-245 degrees Fahrenheit.

5. The method operable to produce the liquid suitable for utilization in an electronic cigarette as recited in claim 4, wherein the step of applying heat to the—mixture of the carrier base and active ingredient is executed for a time range between 5 and 120 minutes.

6. The method operable to produce the liquid suitable for utilization in an electronic cigarette as recited in claim 5, wherein the step of applying ultrasonic frequency to the second vessel having the mixture of the carrier base and active ingredient disposed therein occurs for a time range ranging between 3 and 30 minutes.

7. The method operable to produce the liquid suitable for utilization in an electronic cigarette as recited in claim 6, wherein the active ingredient being selected from cannabis or hemp extract compounds include one of the following: THC, THCV,THCA,CBD,CBT,CBTC,CBCa,CBC,CNB,CBNA,CBDA,CBG,CBGA,HHC,CBDVA, CBGVA,CBDV or CBCV.

8. A liquid formulation suitable for utilization in an electronic cigarette wherein the formulation comprises:
a carrier base, said carrier base being a mixture of propylene glycol and vegetable glycerin;
an active ingredient, wherein said active ingredient is selected from one of the following: cannabis, hemp extract, plant extract or fungi extract.

9. The liquid formulation suitable for utilization in an electronic cigarette as recited in claim 8, wherein the active ingredient being selected from cannabis or hemp extract compounds include one of the following: THC, THCV,THCA,CBD,CBT,CBTC,CBCa,CBC,CNB,CBNA,CBDA,CBG,CBGA,HHC,CBDVA, CBGVA,CBDV or CBCV.
